(19) United States
(12) Patent Application Publication
(10) Pub. No.: US 2022/0202341 A1
(43) Pub. Date: Jun. 30, 2022

(54) System and Method for Detecting Pacer Spikes to Determine a Paced Rhythm in ECGs

(71) Applicant: Tricog Health Pte Ltd, Singapore (SG)

(72) Inventors: Raviteja Upadrashta, Bangalore (IN); Manish Kulariya, Nagaur (IN)

(21) Appl. No.: 17/604,186

(22) PCT Filed: Apr. 15, 2020
(86) PCT No.: PCT/SG2020/050233

§ 371 Date: Oct. 15, 2021

(30) Foreign Application Priority Data
Apr. 16, 2019 (IN) 201941015293

(51) Int. Cl. A61B 5/347; A61B 5/00

(52) U.S. Cl. CPC A61B 5/347; A61B 5/7225; A61B 5/7217

(57) ABSTRACT

The invention provides a system and method for detecting a paced rhythm in a twelve lead ECG. A high-pass filter receives a signal from an ECG lead and pacer spikes within the high-pass filtered signal are distinguished from noise by setting certain threshold limits. Subsequently, clusters of pacer spikes are detected and raw pacer spikes corresponding to each cluster are determined. A pruning process is performed to identify one pacer spike corresponding to each paced beat and raw pacer spike with largest amplitude within a cluster is retained and other pacer spikes within the cluster are eliminated. Further, potential pacer spikes in the ECG lead are determined by eliminating pacer spikes in sections with missing data or high amount of noise. Further post-processing steps are performed to declare the identification of the paced rhythm in the ECG lead.

### CROSS-REFERENCE TO RELATED APPLICATIONS

This application is the United States national phase of International Application No. PCT/SG2020/050233 filed Apr. 15, 2020, and claims priority to Indian Patent Application No. 201941015293 filed Apr. 16, 2019, the disclosures of which are hereby incorporated by reference in their entirety.

### BACKGROUND OF THE INVENTION

### Field of the Invention

The present invention relates to a system and method for detecting a paced rhythm in a twelve lead ECG. More specifically, the invention relates to an identification of potential pacer spikes in the ECG leads by distinguishing the pacer spikes from noise in the ECG lead signals.

### Description of Related Art

Heart diseases have become one of the major causes of death around the world. A proper system or method is necessary to accurately determine a patient's cardiac condition. Abnormal heart rhythms or arrhythmias cause a heart to either beat too slowly or to miss beats. In such cases, the patient is fitted with a pacemaker which is a small electrical device used to treat abnormal heart rhythms. The pacemaker transmits electrical impulses to stimulate contractions of the heart and thus maintain the heartbeat stay within a normal range. The pacemaker can control a heart rhythm to coordinate the contractions of the chambers of the heart.

Electrocardiograms detect abnormal heart rhythms and graphically represent electrical activity of the heart. An electrocardiogram is recorded from a body using a number of electrodes placed in specific predefined areas. An electrocardiogram signal is comprised of very low frequency signals having a P wave, a QRS complex and a T wave. A cardiac abnormality is indicated when there is a deviation in any of the P wave, QRS complex and T wave. Additionally, unwanted noise is also present in the electrocardiogram, however, the noise has to be removed while analyzing electrocardiograms. Electrode contact noise, power-line interference, and muscle noise, etc. can introduce errors into the original electrocardiogram signal. Therefore, it is much needed to reduce the noise in the electrocardiogram signal.

The presence and effect of the pacemaker must be detected by a cardiologist during electrocardiogram (ECG) testing in patients with an implanted pacemaker. Pulses from the pacemaker appear as extremely narrow spikes. In an electrocardiogram, a paced rhythm is difficult to detect in the presence of noise. Additionally, the amplitude of the pacer spikes also varies across different leads in the ECG. The pacer spikes in some leads have maximum amplitude, the other leads have pacer spikes with minimum amplitude. Multiple pacer spikes can also appear around a paced beat.

In conventional methods, pacer spikes across every lead are combined to produce a final list of pacer spikes. However, successful detection of a paced rhythm is difficult from the final list of combined pacer spikes due to the presence of noise.

Thus, in light of the foregoing examination, there is a long-felt need to have a system and method for successfully identifying paced rhythm in a twelve lead ECG by distinguishing the pacer spikes from noise.

### SUMMARY OF INVENTION

The principal object of the invention is to provide a system and method for detecting paced rhythm in a twelve lead ECG.

It is another object of the invention to provide a method for identifying potential pacer spikes in the ECG leads.

It is yet another object of the invention to provide a method for distinguishing the potential pacer spikes from noise in signals of the ECG leads.

It is still another object of the invention to provide a high-pass filter.

It is a further object of the invention to provide threshold limits for identifying the pacer spikes.

It is another object of the invention to provide a method for pruning clusters of the pacer spikes to identify the pacer spikes corresponding to each paced beat.

It is still another object of the invention to provide a method for performing post processing steps to detect the paced rhythm in the twelve lead ECG.

It is a further object of the invention to provide a composite lead signal by overlapping the signals from ECG leads.

### BRIEF DESCRIPTION OF THE DRAWINGS

This invention is illustrated in the accompanying drawings, throughout which, like reference letters indicate corresponding parts in the various figures.

The embodiments herein will be better understood from the following description with reference to the drawings, in which:

FIG. 1 depicts a system illustrating an exemplary system for detecting pacer spikes to determine a paced rhythm, in accordance with an embodiment of the invention.

FIG. 2 depicts an exemplary system for generating a composite signal comprising pacer spikes occurring in multiple ECG leads, in accordance with an embodiment of the invention.

FIG. 3 depicts a graphical representation illustrating successful identification of pacer spikes, in accordance with an embodiment of the invention.

FIG. 4 depicts a flow chart illustrating a method of identifying a paced rhythm in an ECG lead, in accordance with an embodiment of the invention.

FIG. 5 depicts a flowchart illustrating a method of determining whether an ECG lead is a paced ECG lead, in accordance with an embodiment of the invention.

FIG. 6 depicts a flowchart illustrating in detail, a method for identifying a paced rhythm in a twelve lead ECG, in accordance with an embodiment of the invention.

### DESCRIPTION OF THE INVENTION

The present invention discloses a system and method for determining a paced rhythm from potential pacer spikes identified in a twelve lead ECG. A signal from an ECG lead is passed through a high-pass filter. Pacer spikes in the high-pass filtered signal appear as a narrow peak signal occurring between two valleys. A threshold limit is set to distinguish the pacer spikes from noise in the high-pass filtered signal. The identified pacer spikes contain noise and the paced rhythm is hard to distinguish at this stage. Therefore, clusters of the pacer spikes are detected around each beat by setting a threshold limit. Pacer spikes belonging to different clutters are determined by analyzing distances between two adjacent pacer spike locations. A pruning process is performed to identify one pacer spike corresponding to each paced beat. The pacer spike with the largest amplitude within a cluster is retained and other pacer spikes within the cluster are eliminated. Subsequently, sections in the ECG lead with missing data or high amount of noise are identified and the pacer spikes detected within the sections are eliminated, thereby determining potential pacer spikes in the ECG. Post processing steps are performed to declare the identification of the paced rhythm in the ECG.

The embodiments herein and the various features and advantageous details thereof are explained more fully with reference to the non-limiting embodiments that are illustrated in the accompanying drawings and/or detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments herein below provide the details of a system and method for detecting a paced rhythm in a twelve lead ECG. A signal from an ECG lead is passed through a high-pass filter. The high-pass filtered signal contains pacer spikes that appear as a narrow signal peak between two valleys. The pacer spikes are distinguished from noise in the high-pass filtered signal by making a comparison of the distance between two valleys and a threshold limit. The pacer spikes are identified only if the distance of the valleys is smaller than the threshold limit and in case the slope of the sides of the peak signals is above the threshold limit. At this stage, it is difficult to accurately detect a paced rhythm. Subsequently, a cluster of paced spikes is identified around each beat. Thereafter, a pruning process is performed on each cluster to identify one pacer spike corresponding to each paced beat. The pacer spike with largest amplitude within a cluster is retained and all other pacer spikes within the cluster are eliminated. Thus, one pacer spike is identified for each cluster.

Consequently, one or more noisy sections in the ECG lead with missing data and/or high amount of noise are identified and the pacer spikes within the identified sections are eliminated, thereby determining potential pacer spikes in the ECG. One or more post processing steps are carried out to determine a regularity of the pacer spikes, a number of leads in which the pacer spikes are reliably identified, and a set of precordial leads that reliably capture the paced spikes. Upon confirming all the processing steps, a successful detection of a paced rhythm is declared. Thereafter, signals from all the ECG leads are overlapped to form a single composite lead signal to identify the possibility or location of the pacer spikes in all areas including the eliminated areas.

Referring now to the drawings, where similar reference characters denote corresponding features consistently throughout the figures, there are shown preferred embodiments.

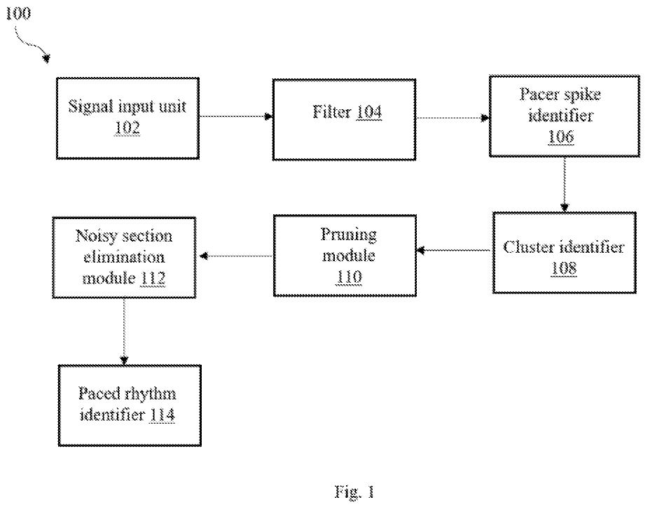

FIG. 1 depicts a system 100 for detecting pacer spikes to determine a paced rhythm, in accordance with an embodiment of the invention.

A signal from an ECG lead is received through a signal input unit 102. Thereafter, the received signal is passed through a high-pass filter 104. The filtered signal is passed through a pacer spike identifier 106, where certain thresholds are used to distinguish one or more pacer spikes from noise in the ECG lead. After the pacer spikes have been identified, a cluster identifier 108 is used to identify one or more clusters of pacer spikes, based on differences in adjacent spike locations. Further, a pruning module 110 is used to identify one pacer spike in each cluster, which corresponds to one beat. Thereafter, noisy sections in the ECG lead are identified and eliminated using a noisy section elimination module 112. Subsequently, a paced rhythm identifier 114 is used to detect an accurate paced rhythm in the ECG lead.

In an embodiment, the number of noisy leads is checked by using a threshold T1 (not shown in figure). In case multiple noisy leads are present, the determination of the paced beat may be terminated.

In a preferred embodiment, the pacer spikes appear in the form of narrow signal peaks, which comprise a signal appearing in a valley-peak-valley pattern. The high-pass filtered signal comprises pacer spikes and noise, where the pacer spikes in the high-pass filtered signal appear as a narrow signal peak occurring between two valleys. The pacer spikes can be distinguished from noise by setting certain threshold limits.

In an embodiment, the system 100 comprises threshold limits T2, T3 and T4. One or more pacer spikes in the ECG lead signal are sampled to identify one or more valley-peak-valley sequences or patterns from the narrow signal peak. An average amplitude of a set of samples S1 is compared to an average amplitude of another set of samples S2 and S3, where the set of samples S2 and S3 occur on either side of the samples S1. After the comparison, in case the amplitude of the set of samples S1 is lower than the set of samples S2 and S3, then the set of samples S1 is classified as a “valley”. On the other hand, in case the amplitude of the set of samples S1 is higher than the set of samples S2 and S3, then the set of samples S1 is classified as a “peak”. In case the amplitude of S2 is higher and the amplitude of S3 occurring at other side of the set of samples S1 is lower, the set of samples S1 is classified as “other”. As a result of the process, all sequences of “valley-peak-valley” patterns are obtained. The pattern obtained from the result is compared with certain thresholds T2, T3, and T4. The thresholds correspond to conditions with which the pacer spikes are identified, as stated below:
a. A fall amplitude occurring between two slopes of the resulting pattern must be greater than the threshold T2.
b. A rise amplitude of the slope must be greater than the threshold T3.
c. A distance between two valleys of the pattern must be lesser than the threshold T4.

With respect to FIG. 1, the identification of clusters of the pacer spikes is accomplished by the cluster identifier 108. As it is difficult to distinguish a paced rhythm in the signal due to the presence of noise in the signal, further processing of the signal is carried out to identify the paced rhythm.

In an embodiment, the clusters of pacer spikes are detected around each paced beat by comparing a distance between adjacent pacer spike locations with a threshold limit T5. In case the distance between two pacer spike locations is greater than the threshold T5, the pacer spikes are considered to belong to different clusters. On the other hand, in case the distance between two pacer spike locations is lesser than the threshold T5, the pacer spikes are considered to belong to the same cluster.

In an embodiment, after identifying the clusters of pacer spikes, the pruning module 110 prunes each cluster to identify one pacer spike corresponding to each paced beat. The pruning is accomplished by retaining one pacer spike with the largest amplitude within a cluster, and eliminating other pacer spikes within the cluster.

In an embodiment, sections with missing data and/or a high amount of noise in the ECG lead are identified. Subsequently, the pacer spikes within the detected noisy sections are eliminated by using the noisy section elimination module 112. Consequently, the paced rhythm identifier 114 determines the presence of a paced rhythm in the ECG signal, as well as identifying potential pacer spikes in the ECG signal.

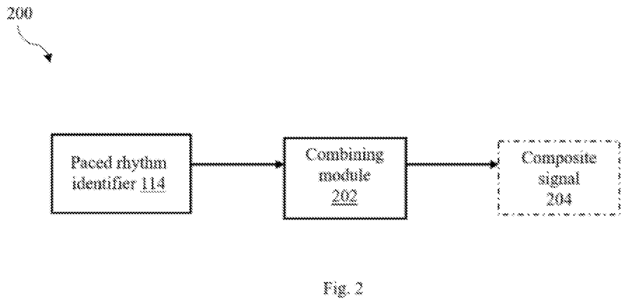

FIG. 2 illustrates an exemplary system 200 for generating a composite signal 204 comprising pacer spikes occurring in multiple ECG leads. The composite signal 204 can be used to determine the possibility or location of the pacer spikes throughout all ECG signals, including eliminated sections.

In an embodiment, each ECG signal comprises pacer spikes and one or more voids. The voids are sections of the signal where noisy sections have been eliminated.

In an embodiment, the signals from multiple ECG leads are combined to create a single composite signal by using a combining module 202. As an example, multiple ECG signals are received from the paced rhythm identifier 114. The combining module 202 combines the ECG signals such that they overlap with respect to time. At any given ‘x’ second, some ECG leads may comprise pacer spikes and other ECG leads may comprise voids. On combining the multiple ECG leads, a composite lead signal 204 is formed, where every ECG lead is overlapped to form the composite signal 204. The composite signal 204 at ‘x’ second comprises one or more pacer spikes, along with any voids that may be present at the ‘x’ second. Thus, even if one ECG lead has a void at ‘x’ second, the presence of a pacer spike on another ECG lead at the same ‘x’ second results in the presence of a pacer spike at ‘x’ second in the composite signal 204.

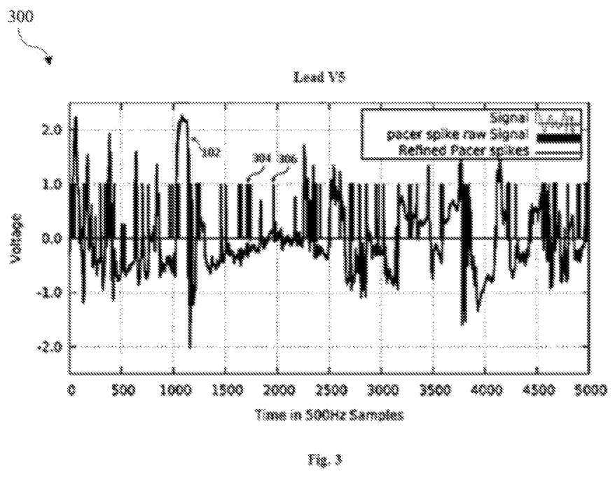

FIG. 3 depicts a graphical representation 300 illustrating a successful identification of pacer spikes despite noises in an ECG lead. The graph is plotted with time in 500 Hz samples (frequency) in x-axis against voltage in Y-axis. The signal from the ECG lead contains pacer spikes that are distinguished from noise present in the ECG lead to successfully identify pacer spikes.

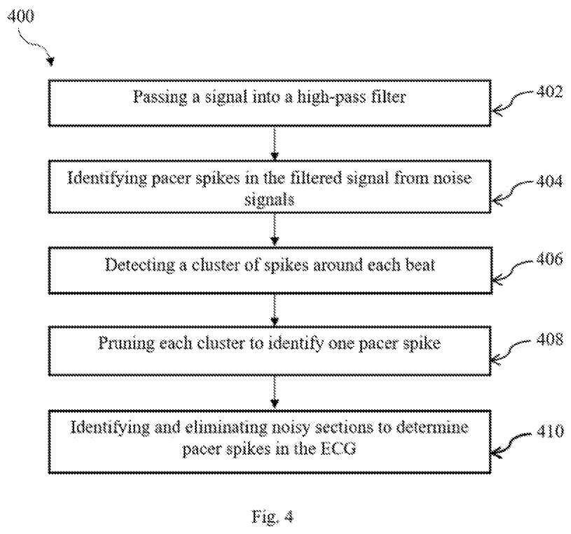

FIG. 4 depicts a flow chart illustrating a method 400 for identifying a paced rhythm in a twelve lead ECG. The method 400 starts at step 402 by passing the ECG lead into a high-pass filter. The high-pass filtered signal coming out from the high pass filter comprises pacer spikes as well as noise. At step 404, the pacer spikes are identified and distinguished from noise in the high-pass filtered signal. The pacer spikes in the high-pass filtered ECG lead appear as a narrow signal peak occurring between two valleys. Threshold limits T2, T3 and T4 are set to reliably distinguish pacer spikes from the noise in the high-pass filtered signal. Subsequently, the pacer spikes are identified by comparing features of the ECG lead with the set threshold limits.

After successful detection of the pacer spikes in the high-pass filtered ECG lead, at step 406, clusters of the pacer spikes are detected. A threshold limit T5 is set to identify the clusters of the pacer spikes. A comparison is made between distances of adjacent pacer spike locations with the threshold limit T5. In case the distance between two adjacent pacer spikes locations is large, the pacer spikes are determined to belong to different clusters. Further, a pruning process is carried out to identify one pacer spike corresponding to each paced beat, by determining one pacer spike for each cluster, as depicted at step 408. The pacer spike with the largest amplitude within each cluster is retained and other pacer spikes within the cluster are eliminated. Subsequently, as depicted at step 410, noisy sections corresponding to missing data and/or a high amount of noise in the ECG lead are further identified. Thus, after eliminating noisy areas, potential pacer spikes in the ECG lead are accurately identified.

In an embodiment, after the identification of the potential pacer spikes, one or more post processing steps are performed. A regularity of the potential pacer spikes, a number of leads that reliably identified the potential pacer spikes, and a presence of precordial leads among the leads that reliably identified the potential pacer spikes are checked in the post processing steps. Thereafter, upon executing and confirming the post processing steps, the paced rhythm is accurately identified.

In one embodiment, the number of pacer spikes detected and a measure of the regularity in which they appear may be used to determine whether a lead is reliable. Additionally, the mean and variance of spike differences may be used as an indicator of the regularity of spike differences.

In one embodiment, at least three ECG leads are required to reliably capture the paced spikes. Subsequently, the declaration of the paced rhythm is made upon confirming the presence of at least two precordial leads among the three leads.

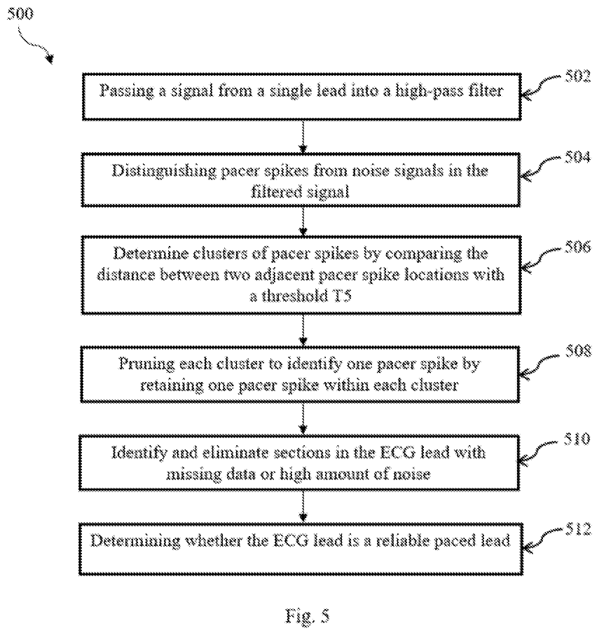

FIG. 5 depicts a method 500 of determining whether an ECG lead is a paced ECG lead. The ECG lead is passed into a high-pass filter, as depicted at step 502. Subsequently, raw pacer spikes are distinguished from noise signals in the high-pass filtered signal, as depicted at step 504. The high-pass filtered signal comprises noise and pacer spikes that appear as a narrow signal peak between two valleys. All sequences of “valley-peak-valley” patterns are obtained from the filtered signal. The pattern obtained from the result is compared with thresholds T2, T3 and T4. A fall amplitude occurring between two slopes of the resulting pattern must be greater than the threshold T2. A rise amplitude of the slope must be greater than the threshold T3. Subsequently, a distance between two valleys of the pattern must be lesser than the threshold T4.

Further, after identifying raw pacer spikes, clusters of the pacer spikes are identified by comparing the distance between adjacent spike locations with a threshold T5, as depicted at step 506. In case the distance between two pacer spike locations is greater than the threshold T5, those pacer spikes are considered as belonging to different clusters. In case the distance between two pacer spike locations is lesser than the threshold T5, pacer spikes are considered as belonging to the same cluster. Subsequently, a pruning process is carried out at each cluster to identify one pacer spike corresponding to each paced beat. The pacer spike in each cluster with a largest amplitude is retained and other pacer spikes in the cluster are eliminated, as depicted at step 508. Subsequently, at step 510, sections with high noise and/or missing data in the ECG lead are identified and eliminated. Thus, potential pacer spikes in the ECG lead are determined. Thereafter, as depicted at step 512, the method determines whether the ECG lead is a reliable paced lead. The number of pacer spikes detected and a measure of the regularity in which they appear may be used to determine whether a lead is reliable. Subsequently, the method 500 concludes that a reliable paced rhythm has been detected.

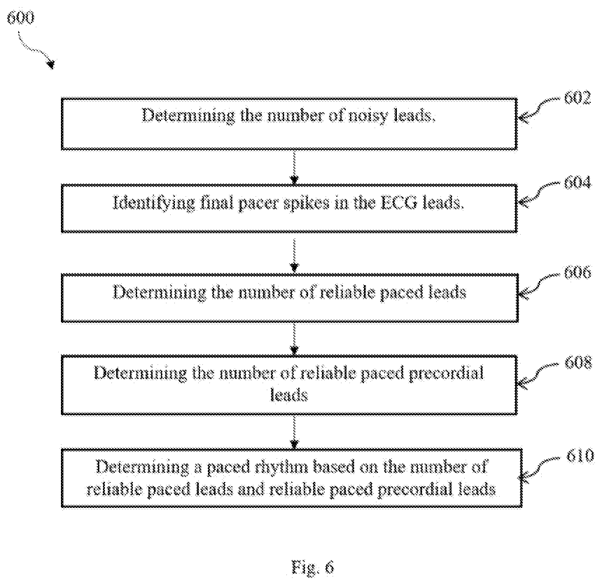

FIG. 6 depicts a detailed method 600 for identifying a paced rhythm in a twelve lead ECG, according to an embodiment. The method comprises determining a number of noisy leads by comparing each ECG lead with a threshold T1, as depicted at step 602. In case the number of noisy leads is not greater than the threshold T1, the method 500 is used to determine whether the ECG lead comprises a reliable paced lead. Subsequently, final pacer spikes are identified in the ECG leads, as depicted at step 604. Thereafter, post processing steps are performed to identify the number of reliable paced leads, as depicted at step 606. In order to determine whether a paced ECG lead is a reliable paced lead, the number of pacer spikes in the ECG lead is compared with a threshold T6. Subsequently, a variance of inter-spike distance is compared with a threshold T7.

Upon confirming that the identified potential pacer spikes are obtained from reliable leads, it is determined whether the reliable paced leads are reliable paced precordial leads, as depicted at step 610. Subsequently, the identification of a paced rhythm is declared upon confirming the presence of precordial paced leads.

In an embodiment, the paced rhythm is detected in case there are at least T8 detected reliable ECG leads, and in case at least T9 among the T8 reliable ECG leads are precordial leads. In a preferred embodiment, ECG leads that reliably detected the potential pacer spikes are checked to identify whether at least three leads reliably captured the potential pacer spikes and whether at least two of those leads are precordial leads.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A system for detecting pacer spikes to determine a paced rhythm in ECG leads, said system comprising:
a filter for filtering an ECG lead;
a pacer spike identifier to identify pacer spikes in the filtered ECG lead;
a cluster identifier to determine one or more clusters of pacer spikes in the ECG lead;
a pruning module to determine one pacer spike corresponding to each cluster of pacer spikes;
a noisy section elimination module for identifying and eliminating pacer spikes in noisy sections in the ECG lead; and
a paced rhythm identifier to determine whether the ECG lead comprises a paced rhythm.

2. The system as claimed in claim 1, wherein the pacer spike identifier identifies pacer spikes by comparing one or more sequence of valley-peak-valley in the filtered signal with one or more thresholds, wherein a fall amplitude occurring between two slopes of the sequence must be greater than a threshold T2, wherein a rise amplitude of the slope must be greater than a threshold T3, and wherein a distance between two valleys of the sequence must be lesser than a threshold T4.

3. The system as claimed in claim 2, wherein clusters of the pacer spikes are detected by comparing a distance between adjacent spike locations with a threshold T5, wherein pacer spikes are determined to belong to a same cluster in case the distance is lesser than T5, and wherein pacer spikes are determined to belong to different clusters in case the distance is greater than T5.

4. The system as claimed in claim 1, wherein the system determines whether the ECG lead is a reliable paced lead based on one or more thresholds, wherein a number of the pacer spikes in the ECG lead is compared with a threshold T6, and wherein a variance of inter-spike distance is compared with a threshold T7.

5. The system as claimed in claim 4, wherein the paced rhythm is detected in case there are at least T8 detected reliable ECG leads and in case at least T9 among the T8 reliable ECG leads are precordial leads.

6. The system as claimed in claim 4, wherein multiple detected ECG leads with paced rhythms are combined to form a composite lead signal to determine locations of the pacer spikes in a twelve lead ECG.

7. A method for detecting pacer spikes to determine a paced rhythm in ECG leads, said method comprising:
passing an ECG lead into a filter;
distinguishing pacer spikes from noise signals in the filtered ECG lead, by using a pacer spike identifier;
detecting one or more clusters of pacer spikes around each paced beat, by using a pacer spike identifier;
pruning the detected one or more clusters to identify one pacer spike corresponding to each paced beat, by using a pruning module;
identifying sections in the ECG lead with missing data or high amount of noise;
eliminating a pacer spike detected in the identified sections, by using a noisy section elimination module;
determining potential pacer spikes in the ECG lead; and
determining whether the ECG lead comprises a paced rhythm, by using a paced rhythm identifier.

8. The method as claimed in claim 7, wherein the method comprises identifying pacer spikes by comparing one or more sequence of valley-peak-valley in the filtered signal with one or more thresholds by using a pacer spike identifier 106, wherein a fall amplitude occurring between two slopes of the sequence must be greater than a threshold T2, wherein a rise amplitude of the slope must be greater than a threshold T3, and wherein a distance between two valleys of the sequence must be lesser than a threshold T4.

9. The method as claimed in claim 7, wherein the method comprises detecting clusters of the pacer spikes by comparing a distance between adjacent spike locations with a threshold T5, wherein pacer spikes are determined to belong to a same cluster in case the distance is lesser than T5, and wherein pacer spikes are determined to belong to different clusters in case the distance is greater than T5.

10. The method as claimed in claim 9, wherein the method comprises determining whether the ECG lead is a reliable paced lead based on one or more thresholds, wherein a number of the pacer spikes in the ECG lead is compared with a threshold T6, and wherein a variance of inter-spike distance is compared with a threshold T7.

11. The method as claimed in claim 10, wherein the method comprises detecting the paced rhythm in case there are at least T8 detected reliable ECG leads and in case at least T9 among the T8 reliable ECG leads are precordial leads.

12. The method as claimed in claim 10, wherein multiple detected ECG leads with paced rhythms are combined to form a composite lead signal to determine locations of the pacer spikes in a twelve lead ECG.
